(19) United States
(12) Patent Application Publication
(10) Pub. No.: US 2015/0265526 A1
(43) Pub. Date: Sep. 24, 2015

(54) TOPICAL COMPOSITION FOR THE COSMETIC TREATMENT OF THE HUMAN SKIN

(71) Applicant: Beaute Pacifique ApS, Hadsund (DK)

(72) Inventor: Flemming Kjaergaard CHRISTENSEN, Hadsund (DK)

(73) Assignee: Beaute Pacifique ApS, Hadsund (DK)

(21) Appl. No.: 14/219,115

(22) Filed: Mar. 19, 2014

(51) Int. Cl. A61K 8/97; A61K 8/34; A61Q 1/00; A61K 8/67; A61Q 19/00; A61K 8/31; A61K 8/49

(52) U.S. Cl. CPC A61K 8/97; A61K 8/31; A61K 8/347; A61K 8/498; A61K 8/678; A61Q 19/00; A61Q 1/00; A61K 2800/5922

(57) ABSTRACT

A topical composition for the cosmetic treatment of the human skin and of the type comprising a vehicle in the form of squalane and high contents of active anti-oxidative substances, which composition comprises Chilean Grape Seed Extract (GSPE) in the form of a cream or a serum solution and comprises said active anti-oxidative substances in the form of resveratrol and procyanidin.

The use of a topical composition according to any of the proceeding claims where one or more drops of said serum solution or a small amount of said cream is applied to the skin by way of example the skin of the back of a hand and is mixed with a suitable amount of make-up, which make-up mixture afterwards is applied to the skin of the face.

### FIELD OF THE INVENTION

The present invention relates to a topical composition for the cosmetic treatment of the human skin and of the type as indicated in the preamble til claim 1.

Furthermore, the present invention relates to the use of such topical composition in relation to the preparation of make-up for use on the human skin.

### BACKGROUND OF THE INVENTION

Usually, the compositions for cosmetic treatment of the human skin, such as wrinkled skin, comprise lotions which according to the instructions of the products are to be applied onto the skin, for example the skin of the face or onto other parts of the body, where the expected effect is desired.

### OBJECT OF THE INVENTION

The present invention has for its purpose to indicate a new and improved topical composition of the initial mention type, and by means of which may be achieved prompt correction of an imperfect skin surface and other fast and long term benefits.

### DESCRIPTION OF THE INVENTION

The topical composition for the cosmetic treatment of the human skin of the type as indicated above is characterized in comprising Chilean Grape Seed Extract (GSPE) in the form of a cream or a serum solution and comprising said active anti-oxidative substances in the form of resveratrol and procyanidin.

Appropriately, said topical composition according to the invention is furthermore characterized in that said vehicle also comprises vitis vinifera in the form of grape seed oil extract.

Advantageously, said topical composition according to the invention is furthermore characterized in that said vehicle also comprise tocopheryl acetate and/or tocophenol (vitamin E).

Said fast benefits are prompt correction of an imperfect skin surface, adds a visible glow-effect to the make-up sand helps the make-up to camouflage uneven pigmentation.

Additionally the following long term benefits are achieved: The contents of a sophisticated extract from Chilean Andes mountains grape seeds (GSPE) diminishing excessive rednesses, i.e. Rosacea looking skin, while the contents of resveratrol and procyanidin provide anti-oxidative defence against free radicals and reduce ageing signs. The contents of skin-compatible squalane improve the submerging of the ingredients into the skin and replenish what is lost in sun-damaged and aged skin. Additionally, said composition contains two types of vitamin E.

The present invention furthermore relates to the use of said topical composition according to any of the proceeding claims making use of one or more drops of said serum solution, which is applied to the skin by way of example the skin of the back of a hand and is mixed with a suitable amount of make-up, which make-up mixture afterwards is applied to the skin of the face.

Alternatively, the use of said such topical composition according to any of the proceeding claims makes use of a small amount of said cream, which is applied to the skin by way of example the skin of the back of a hand and is mixed with a suitable amount of make-up, which make-up mixture afterwards is applied to the skin of the face.

### DETAILED DESCRIPTION OF THE INVENTION

The submersive serum according to the present invention is based on new medical discoveries relating to a sophisticated Chilean Grape Seed Extract (GSPE). These grapes are grown at hill sides in the Andes Mountains where the sun's UV-radiation is very severe. During the evolution these special grapes have adapted to the extreme UV-exposure and secured survival of the genes. The grapes have developed their own defence against free radicals built on high contents of the two active anti-oxidative substances resveratrol and procyanidin. Such submersive serum provides to the human skin the same active ingredients as what gave survival of the fittest grapes. The action of the GSPE with resveratrol and procyanidin is at the same time both anti-age and anti-inflammatory and blended with squalane, the serum goes submersive. Said serum is a new first step to a perfect looking make-up. The serum fills up the pits and the pores and an imperfect skin surface and enables the make-up to also camouflage uneven pigmentation.

### EXAMPLE 1

Said topical composition may consist of the following ingredients:

| Ingredient | content by weight % |
|---|---|
| Squalane (based on olive oil) | 50-80 |
| Vitis vinifera grape seed oil extracts | 18-48 |
| Tocopherol (vitamin E) | 0.2-1.5 |
| Tocopheryl Acetate (vitamin E) | 0.5-1.8 |

### EXAMPLE 2

Said topical composition may consist of the following ingredients:

| Ingredient | content by weight % |
|---|---|
| Squalane (based on olive oil) | 76.50 |
| Vitis vinifera grape seed oil extracts | 22.00 |
| Tocopherol (vitamin E) | 0.50 |
| Tocopheryl Acetate (vitamin E) | 1.00 |

Chilean vitis vinifera grape seed oil extracts (GSPE) has quite special qualities because the grape seeds have shown to be UV-resistant such that the DNA or genes thereof are not infected by the very severe UV-radiation from the sun at the hill sides in the Andes Mountains.

The basic ingredient of the vehicle of the topical composition is squalane (based on olive oil) and the function of the two vitamin E ingredients tocopherol and tocopheryl acetate is that both ingredients provide anti-oxidative defence against free radicals and reduce aging signs of the skin—additionally tocopheryl acetate has also an anti-rancid effect to the vehicle of the topical composition.

1. A topical composition for the cosmetic treatment of the human skin and of the type comprising a vehicle in the form of squalane and high contents of active anti-oxidative substances characterized in comprising Chilean Grape Seed Extract (GSPE) in the form of a cream or a serum solution and comprising said active anti-oxidative substances in the form of resveratrol and procyanidin.

2. Topical composition according to claim 1, characterized in that said vehicle further comprising vitis vinifera in the form of grape seed oil extract.

3. Topical composition according to claim 1, characterized in that said vehicle further comprising tocopheryl acetate and/or tocophenol.

4. Use of a topical composition according to claim 1, where one or more drops of said serum solution is applied to the skin by way of example the skin of the back of a hand and is mixed with a suitable amount of make-up, which make-up mixture afterwards is applied to the skin of the face.

5. Use of a topical composition according to claim 1, where a small amount of said cream is applied to the skin by way of example the skin of the back of a hand and is mixed with a suitable amount of make-up, which make-up mixture afterwards is applied to the skin of the face.

6. Use of a topical composition according claim 2, where one or more drops of said serum solution is applied to the skin by way of example the skin of the back of a hand and is mixed with a suitable amount of make-up, which make-up mixture afterwards is applied to the skin of the face.

7. Use of a topical composition according claim 3, where one or more drops of said serum solution is applied to the skin by way of example the skin of the back of a hand and is mixed with a suitable amount of make-up, which make-up mixture afterwards is applied to the skin of the face.

8. Use of a topical composition according to claim 1, where a small amount of said cream is applied to the skin by way of example the skin of the back of a hand and is mixed with a suitable amount of make-up, which make-up mixture afterwards is applied to the skin of the face.

9. Use of a topical composition according to claim 2, where a small amount of said cream is applied to the skin by way of example the skin of the back of a hand and is mixed with a suitable amount of make-up, which make-up mixture afterwards is applied to the skin of the face.

10. Use of a topical composition according to claim 3, where a small amount of said cream is applied to the skin by way of example the skin of the back of a hand and is mixed with a suitable amount of make-up, which make-up mixture afterwards is applied to the skin of the face.

11. Use of a topical composition according to claim 4, where a small amount of said cream is applied to the skin by way of example the skin of the back of a hand and is mixed with a suitable amount of make-up, which make-up mixture afterwards is applied to the skin of the face.

12. Use of a topical composition according to claim 5, where a small amount of said cream is applied to the skin by way of example the skin of the back of a hand and is mixed with a suitable amount of make-up, which make-up mixture afterwards is applied to the skin of the face.

13. Use of a topical composition according to claim 6, where a small amount of said cream is applied to the skin by way of example the skin of the back of a hand and is mixed with a suitable amount of make-up, which make-up mixture afterwards is applied to the skin of the face.
